(19) United States
(12) Patent Application Publication
(10) Pub. No.: US 2025/0359841 A1
(43) Pub. Date: Nov. 27, 2025

(54) DEVICE, NON-TRANSITORY COMPUTER-READABLE STORAGE MEDIUM AND SYSTEM FOR IDENTIFYING BREATHING SOUND

(71) Applicant: Biosono Inc., Sendai-shi (JP)

(72) Inventors: Ken TOYAMA, Sendai-shi (JP); Takeshi KURISAKI, Sendai-shi (JP)

(73) Assignee: Biosono Inc., Sendai-shi (JP)

(21) Appl. No.: 19/215,450

(22) Filed: May 22, 2025

(30) Foreign Application Priority Data
May 27, 2024 (JP) 2024-085346

(51) Int. Cl. A61B 7/00; A61B 7/02; A61B 7/04; H04R 1/46; H04R 3/00

(52) U.S. Cl. CPC A61B 7/003; A61B 7/026; A61B 7/04; H04R 1/46; H04R 3/005

(57) ABSTRACT

A device is configured to identify a breathing sound contained in acoustic signals from outside. The device has a processor, a display coupled to the processor and a memory coupled to the processor and storing instructions. The instructions, when executed by the processor, cause the processor to perform a first identifying process upon a start command and to perform a second identifying process upon a stop command subsequent to the start command. In the first identifying process, a first identified result is displayed on the display, the first identified result indicating whether the breathing sound obtained during a first predetermined period is normal or abnormal. In the second identifying process, a second identified result is displayed on the display, the second identified result indicating whether the breathing sound obtained during a second predetermined period is normal or abnormal.

### CROSS REFERENCE TO RELATED APPLICATIONS

This application is based on and claims priority under 35 U.S.C. § 119 to Japanese Patent Application No. JP 2024-085346 filed May 27, 2024, the content of which is incorporated herein in its entirety by reference.

### BACKGROUND OF THE INVENTION

This invention relates to a device for identifying a breathing sound, or a device configured to detect abnormal breathing caused by events such as eating.

For example, JP2012-205693A (Patent Document 1) discloses a breathing-sound analyzing system configured to detect abnormal breathing caused by an accident such as aspiration during sleep. The content of Patent Document 1 is incorporated herein by reference.

The breathing-sound analyzing system of Patent Document 1 comprises a breathing-sound analyzing device (device for identifying a breathing sound), a microphone and a sensor band for sensing a breathing sound. The microphone and the sensor band are attached to a subject, or a sleeping person. In detail, the microphone is attached on the throat of the subject via adhering, and the sensor band is wrapped around the chest of the subject. The microphone converts collected sounds into electrical signals, namely acoustic signals, and send them to the breathing-sound analyzing device. The sensor band converts motions of the chest of the subject into electrical signals, namely motion signals, and send them to the breathing-sound analyzing device. The breathing-sound analyzing device continues to extract a breathing sound of the subject based on the received acoustic signals and the received motion signals. The breathing-sound analyzing device detects abnormal breathing by comparing a large number of the extracted breathing sounds with each other using a predetermined algorism. The result of the aforementioned detection is displayed on a display of the breathing-sound analyzing device.

The breathing-sound analyzing device of Patent Document 1 can detect abnormal breathing of the subject which is caused by an accident such as invasion of saliva into the larynx. A care person of the subject can notice abnormal breathing of the subject through the display screen without attending the sleeping subject.

Abnormal breathing may occur not only during sleep but also because of various events. For example, abnormal breathing may occur when a piece of food remains in respiratory organs as a result of eating. Moreover, breathing tends to be changed in frequency and depth when a person takes exercise, a bath or medicine such as inhalants for asthma, and thereby abnormal breathing may appear or disappear. Thus, the presence or absence of abnormal breathing may be changed because of various events such as eating, exercising, bathing or taking medicine. Abnormal breathing sometimes suggests abnormality of respiratory organs. It is desirable especially in an aging society to be able to easily detect abnormal breathing which might be caused by various events.

### SUMMARY OF THE INVENTION

It is therefore an object of the present invention to provide a device for identifying a breathing sound, or a device configured to detect abnormal breathing caused by events such as eating.

An aspect of the present invention provides a device for identifying a breathing sound contained in acoustic signals received from outside. The device comprises a processor, a display coupled to the processor and a memory coupled to the processor and storing instructions. The instructions, when executed by the processor, cause the processor to: perform a first identifying process upon a start command; perform a second identifying process upon a stop command subsequent to the start command; display a first identified result on the display in the first identifying process, the first identified result indicating whether a breathing sound obtained during a first predetermined period is normal or abnormal; and display a second identified result on the display in the second identifying process, the second identified result indicating whether a breathing sound obtained during a second predetermined period is normal or abnormal.

The device of an aspect of the present invention can display two identified results including the first identified result for the first predetermined period and the second identified result for the second predetermined period subsequent to the first predetermined period. Accordingly, two identified results before and after an event such as eating can be compared with each other by inputting the start command before the event and inputting the stop command after the event. More specifically, it can be considered that abnormal breathing occurs because of eating if the identified result before the eating of a person is normal and the identified result after the eating of the person is abnormal. For example, it can be considered that a piece of food might remain in an upper respiratory organ of the person because of the eating. Thus, an aspect of the present invention provides a device for identifying a breathing sound, or a device configured to detect abnormal breathing caused by events such as eating.

An appreciation of the objectives of the present invention and a more complete understanding of its configuration may be had by studying the following description of the preferred embodiment and by referring to the accompanying drawings.

### BRIEF DESCRIPTION OF THE DRAWINGS

FIG. 1 is a view showing a system for identifying a breathing sound according to an embodiment of the present invention.

FIG. 2 is a perspective view showing an acoustic sensor of the system of FIG. 1, wherein a position of a lamp accommodated in a body of the acoustic sensor is illustrated with dashed line.

FIG. 3 is an exploded, perspective view showing the acoustic sensor of FIG. 2, wherein a position of the lamp is illustrated with dashed line.

FIG. 4 is a bottom view showing the body of the acoustic sensor of FIG. 3.

FIG. 5 is a front view showing the body of the acoustic sensor of FIG. 3, wherein a part of an outline of a base of the acoustic sensor is illustrated with chain dotted line, a part of the acoustic sensor enclosed by dashed line is enlarged and illustrated, and in the enlarged view, an outline of a hidden recessed portion is illustrated with two-dot chain line.

FIG. 6 is a block diagram showing a device for identifying a breathing sound of the system of FIG. 1.

FIG. 7 is a view showing a structure of acoustic data stored in an auxiliary storage of the device of FIG. 6.

FIG. 8 is a view showing timings at each of which the device of FIG. 6 identifies a breathing sound.

FIG. 9 is a flowchart showing a control process of an analyzer of the device of FIG. 6, wherein the analyzer is a computer program for identifying a breathing sound.

FIG. 10 is a flowchart showing an acoustic-signal memorizing process of the analyzer of FIG. 9.

FIG. 11 is a flowchart showing a first identifying process of the analyzer of FIG. 9.

FIG. 12 is a flowchart showing a second identifying process of the analyzer of FIG. 9.

FIG. 13 is a view for explaining about input and output layers of an abnormality determination model of FIGS. 11 and 12.

FIG. 14 is a view showing an example of the abnormality determination model of FIG. 13, wherein the illustrated example is a convolutional neural network model.

FIG. 15 is a view showing an example of a generating method of the abnormality determination model of FIGS. 11 and 12.

FIG. 16 is a view showing a modification of the system of FIG. 1.

FIG. 17 is a view showing timings at each of which the system of FIG. 16 identifies a breathing sound.

While the invention is susceptible to various modifications and alternative forms, specific embodiments thereof are shown by way of example in the drawings and will herein be described in detail. It should be understood, however, that the drawings and detailed description thereto are not intended to limit the invention to the particular form disclosed, but on the contrary, the intention is to cover all modifications, equivalents and alternatives falling within the spirit and scope of the present invention as defined by the appended claims.

### DETAILED DESCRIPTION

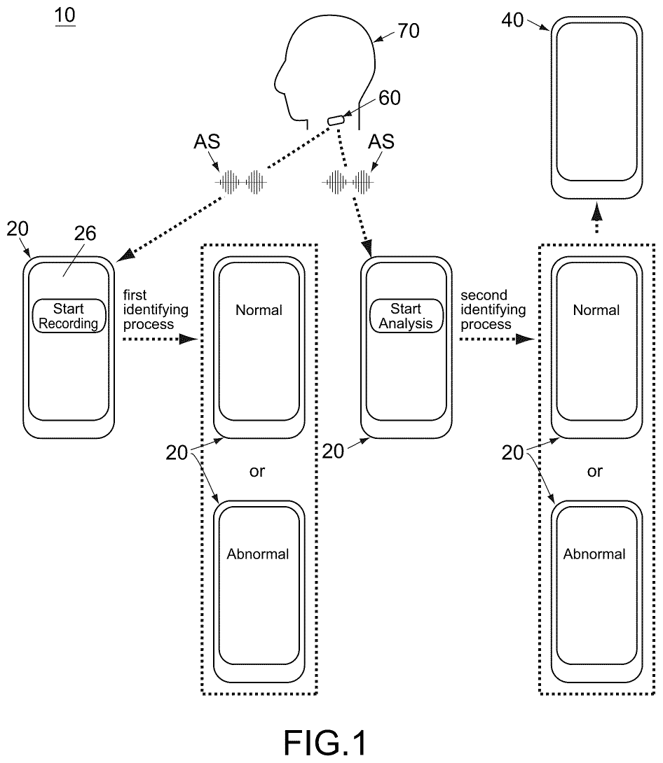

Referring to FIG. 1, a system 10 for identifying a breathing sound according to an embodiment of the present invention is configured to identify a breathing sound of a subject 70 as normal or abnormal. The system 10 of the present embodiment is suitable to be used in a nursing-care facility. The subject 70 of the present embodiment is a senior citizen who lives in a nursing-care facility. However, the present invention is not limited thereto but is variously applicable. For example, the system 10 may be used in a gym or may be used in an average home. For example, the system 10 may be used for home nursing in an average home.

The system 10 of the present embodiment comprises a device 20 for identifying a breathing sound, a management device 40 and an acoustic sensor 60.

The acoustic sensor 60 is a compact electronic device attachable on and detachable from the subject 70. In detail, the acoustic sensor 60 can be attached on a skin of the throat of the subject 70 via adhering. The acoustic sensor 60 comprises various electronic components such as a microphone (not shown). The microphone of the acoustic sensor 60 is configured to collect sounds such as a breathing sound generated in the respiratory tract of the subject 70 and is configured to convert them into electrical signals, namely acoustic signals AS. The acoustic sensor 60 of the present embodiment is configured to send the acoustic signals AS via short-range wireless communication such as Bluetooth.

As describe above, the acoustic sensor 60 of the present embodiment has a sensing function for collecting sounds therearound and converting them into the acoustic signals AS and a sending function for sending the acoustic signals AS via short-range wireless communication. However, the present invention is not limited thereto. For example, the acoustic sensor 60 may further has another function in addition to the sensing function and the sending function. The acoustic sensor 60 may send the acoustic signals AS via a wireless communication other than the short-range wireless communication or send them by wire through a communication cable.

Each of the device 20 and the management device 40 of the present embodiment is a mobile terminal such as a smart phone. Each of the device 20 and the management device 40 is easy to carry. However, the present invention is not limited thereto. For example, at least one of the device 20 and the management device 40 may be an easily carriable personal computer (PC) or may be a desk top PC.

The device 20 is configured to receive the acoustic signals AS. More specifically, the device 20 of the present embodiment is paired with the acoustic sensor 60 and is arranged at a distance from the acoustic sensor 60 so that the device 20 can receive the acoustic signals AS. However, the present invention is not limited thereto. For example, in an instance in which the acoustic signals AS are sent by wire through a communication cable, the device 20 may be connected to the communication cable.

The device 20 is configured to send various data to the management device 40. The management device 40 is configured to receive the data sent from the device 20. The communication method between the device 20 and the management device 40 is not specifically limited.

The device 20 is configured to identify a breathing sound contained in the acoustic signals AS received outside. In detail, the device 20 performs two identifying processes consisting of a first identifying process and a second identifying process subsequent to the first identifying process. In each of the first identifying process and the second identifying process, the device 20 identifies, or classifies, whether the breathing sound of the subject 70 contained in the received acoustic signals AS is normal or abnormal and displays the identified result. The device 20 sends the identified results of the first identifying process and the second identifying process to the management device 40.

In the present embodiment, the device 20 is operated by a caregiver of a nursing-care facility, and the management device 40 is operated by a manager of the nursing-care facility. For example, the device 20 identifies a breathing sound before eating of the subject 70 by the first identifying process and identifies a breathing sound after eating of the subject 70 by the second identifying process. For example, in a case where a breathing sound before eating is normal and a breathing sound after eating is abnormal, the caregiver and the manager can be aware that abnormal breathing has been generated because of the eating and thereby can provide appropriate care for the subject 70. For example, the caregiver can instruct the subject 70 to gargle so that a piece of food is removed from respiratory organs of the subject 70. The manager can consider whether content of meals should be changed or not.

As described above, the operators of the device 20 and the management device 40 of the present embodiment are a caregiver and a manager of a nursing-care facility, respectively. However, the present invention is not limited thereto. For example, the operator of each of the device 20 and the management device 40 may be varied depending on the purpose of the device 20. For example, when the system 10 is used in an average home, a member of the family of the subject 70 may operate the device 20, and one of staffs of a nursing-care facility far from the home may operate the management device 40.

The system 10 shown in FIG. 1 comprises the single device 20, the single management device 40 and the single acoustic sensor 60. However, the present invention is not limited thereto. For example, the system 10 may further comprise another device in addition to the aforementioned devices. The system 10 may comprise two or more of the devices 20 and two or more of the acoustic sensors 60. The system 10 may comprise none of the management device 40.

Hereafter, explanation will be made about the acoustic sensor 60 of the present embodiment.

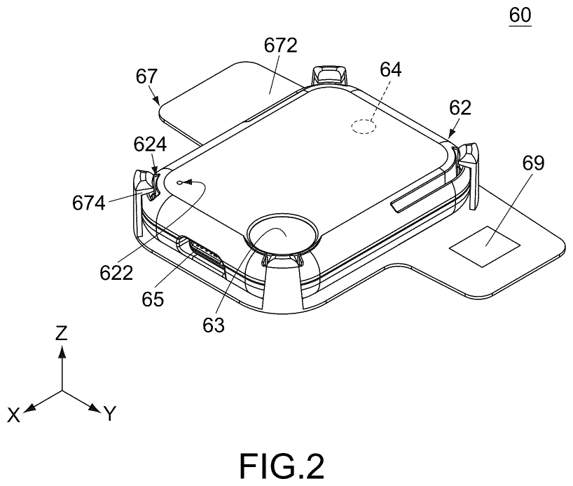

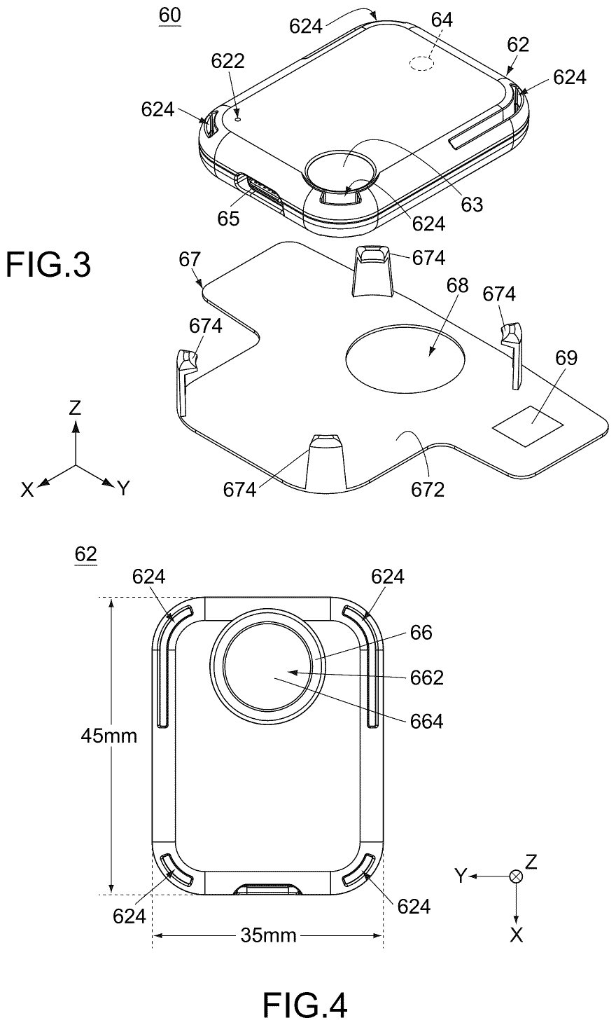

Referring to FIGS. 2 and 3, the acoustic sensor 60 of the present embodiment comprises a body 62 and a base 67. The body 62 is a member which is configured to perform the sensing function and the sending function of the acoustic sensor 60. The base 67 is configured to be attached on a skin of the throat of the subject 70 (see FIG. 1) via adhering.

Referring to FIGS. 3 to 5, the body 62 of the present embodiment has a thin, rectangular flat-plate shape. In detail, the body 62 has a size of about 45 mm in a front-rear direction (X-direction) and has a size of about 35 mm in a lateral direction (Y-direction) perpendicular to the front-rear direction. Moreover, the body 62 has a size of about 8.5 mm in an up-down direction (Z-direction) perpendicular to both the front-rear direction and the lateral direction. The words such as the up-down direction do not indicate the absolute relation relative to the ground but merely indicate a relative relation under a definition that a part of the surface of the body 62 which is configured to face the skin of the subject 70 (see FIG. 1) is a lower surface.

The body 62 of the present embodiment has a switch 63, a lamp 64, a connector 65 and a contact portion 66 in addition to various electronic components such as a microphone (not shown) accommodated in the body 62. The body 62 has an upper surface (positive Z-side surface) and a lower surface (negative Z-side surface). The upper surface of the body 62 is formed with four locked portions 624. The lower surface of the body 62 is formed with a recessed portion 662.

Referring to FIG. 3, the switch 63 is a power switch of the body 62. The switch 63 takes on-state when pushed and takes off-state when pushed again. The body 62 works when the switch 63 is under the on-state. The lamp 64 is accommodated in the body 62 and emits light when the body 62 works. The thus-lit lamp 64 radiates light upward from the body 62 through a half-transparent member. The connector 65 is used when the body 62 is electrically charged.

Referring to FIG. 2 together with FIG. 1, the switch 63 and the lamp 64 of the present embodiment are also used in a pairing process in which the device 20 is paired with the acoustic sensor 60. More specifically, after the switch 63 is kept being pushed for five to seven seconds, the acoustic sensor 60 takes a preparing state, and the lamp 64 flashes in blue and red alternately. When the pairing process comes to end, the acoustic sensor 60 takes a workable state, and the lamp 64 lights in red. However, the present invention is not limited thereto. For example, the condition where the lamp 64 lights or flashes alternately and the color of the lamp 64 can be modified as necessary.

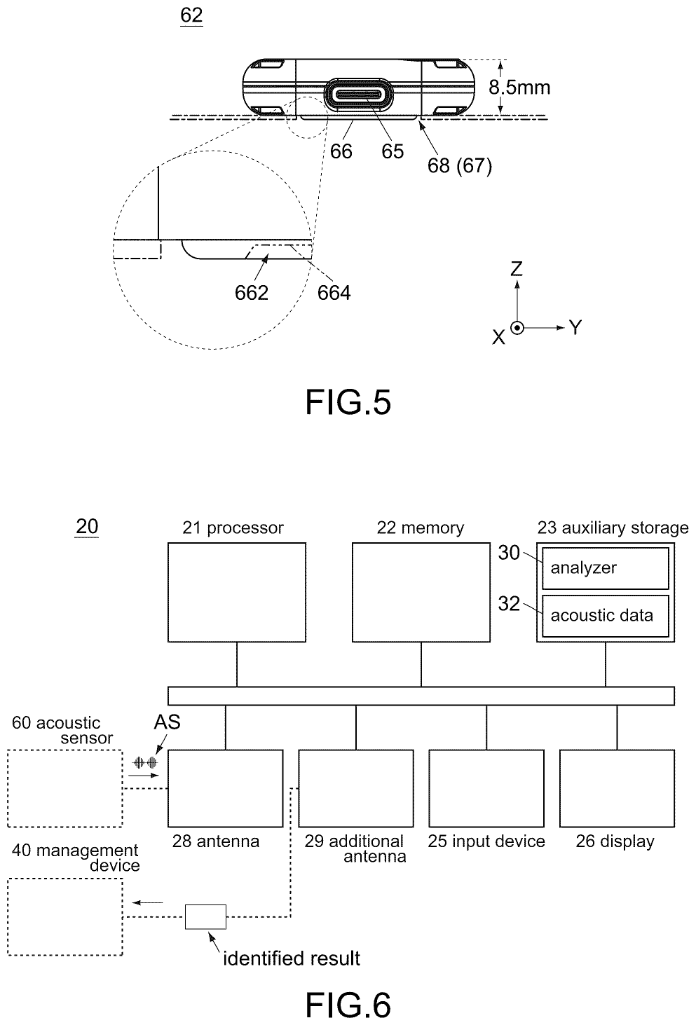

Referring to FIGS. 4 and 5, the contact portion 66 projects downward from the lower surface of the body 62 in the up-down direction and has a ring shape in a horizontal plane (XY-plane) perpendicular to the up-down direction. The contact portion 66 of the present embodiment has a circular ring shape with no gap in the horizontal plane. However, the present invention is not limited thereto. For example, the contact portion 66 may have a circular ring shape with gaps or may have a rectangular ring shape in the horizontal plane.

The recessed portion 662 is recessed upward from the lower surface of the body 62 and is enclosed by the contact portion 66 in the horizontal plane. The recessed portion 662 is provided with a sound collection sheet 664 arranged on an upper part thereof. The sound collection sheet 664 is configured to vibrate when receiving sound. The sound collection sheet 664 of the present embodiment has a circular shape in the horizontal plane and easily vibrates uniformly. However, the present invention is not limited thereto. For example, the shape of the sound collection sheet 664 can be modified as necessary.

The body 62 of the present embodiment has the aforementioned structure. However, the present invention is not limited thereto. For example, the structure of the body 62 can be modified as necessary.

Referring to FIG. 3, the base 67 has four lock portions 674 and a bottom portion 672 which is thin and is easily bent. The bottom portion 672 extends along the horizontal plane and is formed with a passing hole 68. Thus, the base 67 is formed with the passing hole 68. The passing hole 68 has a circular shape corresponding to the contact portion 66 (see FIG. 4) in the horizontal plane and passes through the bottom portion 672 in the up-down direction. The four lock portions 674 enclosed the passing hole 68 in the horizontal plane and projects upward from the bottom portion 672. The base 67 is provided with a tag 69 attached thereto. For example, the tag 69 is printed with an identifier of the acoustic sensor 60 or a two-dimensional matrix barcode corresponding to the identifier.

The base 67 of the present embodiment has the aforementioned structure. However, the present invention is not limited thereto. For example, the structure of the base 67 can be modified as necessary. For example, the tag 69 may be provided as necessary.

Referring to FIGS. 2 and 3 together with FIG. 5, the body 62 is attached to the base 67 so that the contact portion 66 passes through the passing hole 68. The lock portions 674 lock the locked portions 624, respectively, when the body 62 is attached to the base 67. Meanwhile, a lower surface of the contact portion 66 is substantially flash with a lower surface of the bottom portion 672. The sound collection sheet 664 faces the skin of the subject 70 when the bottom portion 672 is attached on the skin of the subject 70 (see FIG. 1) via adhering.

According to the present embodiment, the sound collection sheet 664 faces the skin of the throat of the subject 70 when the base 67 is attached on the skin of the throat of the subject 70 (see FIG. 1) via adhering. The thus-arranged sound collection sheet 664 can reliably transmit breathing sounds generated in the respiratory tract of the subject 70 into the body 62. Moreover, noise such as conversation around the subject 70 is hardly transmitted into the body 62. Accordingly, the acoustic sensor 60 can effectively collect breathing sounds generated in the throat of the subject 70 while reducing noise.

Referring to FIG. 2, the acoustic sensor 60 of the present embodiment comprises a noise cancellation circuit (not shown) and an external microphone 622. The noise cancellation circuit is incorporated in the body 62. The external microphone 622 opens at the upper surface of the body 62 and is connected to the noise cancellation circuit. The external microphone 622 converts noise such as conversation, which occurs about a side of the acoustic sensor 60 opposite to the throat of the subject 70, into signals and transmit them to the noise cancellation circuit. The noise cancellation circuit generates counter-phase signals based on the signals transmitted from the external microphone 622 to cancel the noise. As described above, the acoustic sensor 60 of the present embodiment has a noise cancel function so as to selectively collect breathing sounds generated in the throat of the subject 70.

The acoustic sensor 60 of the present embodiment has the aforementioned structure and can be attached to the subject 70 (see FIG. 1) via adhering as described above. However, the present invention is not limited thereto. For example, the acoustic sensor 60 may be attached to a part of the subject 70 other than a skin of the throat thereof.

Hereafter, explanation will be made about the device 20 (see FIG. 1) of the present embodiment.

Referring to FIG. 6, the device 20 of the present embodiment comprises a processor 21, a memory 22, an auxiliary storage 23, an input device 25, a display 26, an antenna 28 and an additional antenna 29. The processor 21 is communicatively connected with the other devices via an internal bus. In other words, the device 20 comprises the memory 22, the auxiliary storage 23, the input device 25, the display 26, the antenna 28 and the additional antenna 29 each coupled to the processor 21. The illustrated device 20 comprises only the aforementioned devices. However, the present invention is not limited thereto. For example, the device 20 may further comprise another device such as a speaker.

According to the present embodiment, each of the aforementioned devices is a part of a single mobile terminal. For example, the display 26 is a screen of the mobile terminal. The input device 25 is a virtual input portion such as a keyboard and input buttons displayed on the screen of the mobile terminal. The antenna 28 is a near-field communication antenna incorporated in the mobile terminal. The antenna 28 is configured to receive the acoustic signals AS sent from the acoustic sensor 60 paired with the device 20. The additional antenna 29 is a general communication antenna incorporated in the mobile terminal. The additional antenna 29 is configured to send the identified results of a breathing sound of the subject 70 (see FIG. 1) to the management device 40.

The device 20 of the present embodiment has the aforementioned structure. However, the present invention is not limited thereto. For example, in an instance in which the device 20 is a PC, the display 26 may be a liquid crystal display formed separately from the processor 21, and the input device 25 may be a key board or a mouse formed separately from the processor 21. In this instance, each of the input device 25 and the display 26 may be communicatively connected with the processor 21 via a communication cable.

The processor 21 comprises a central processing unit (CPU: not shown). For example, the auxiliary storage 23 is an electrically erasable programmable read-only memory (EEPROM). The auxiliary storage 23 can store various files including acoustic data 32 and an executable file of an analyzer 30 which is a computer program for identifying a breathing sound. The auxiliary storage 23 performs various actions such as retrieval and storing of files in accordance with a command sent from the processor 21. The CPU of the processor 21 gets an executable file stored in the auxiliary storage 23, loads it into the memory 22 and performs various functions by executing instructions memorized in the executable file. Thus, the memory 22 is configured to store instructions executable by the processor 21.

The input device 25 is configured to send input characters and appointed locations and ranges to the processor 21. The display 26 is configured to display characters and images sent from the processor 21. For example, the CPU (not shown) of the processor 21 loads the analyzer 30 into the memory 22 in response to a command input from the input device 25 and executes a process for identifying a breathing sound. Thus, the analyzer 30 is a computer program which is configured to make the mobile terminal work as the device 20. For example, the analyzer 30 is installed in the auxiliary storage 23 via the world wide web (WEB).

As describe above, the CPU (not shown) of the processor 21 executes a computer program such as the analyzer 30 in fact. More specifically, the memory 22 coupled to the processor 21 stores instructions of the analyzer 30. The instructions of the analyzer 30, when executed by the processor 21, cause the processor 21 to perform various processes. However, the explanation described below includes explanation in which a computer program such as the analyzer 30 itself performs processes and explanation in which a process itself of a computer program such as the analyzer 30 executed by the CPU performs processes.

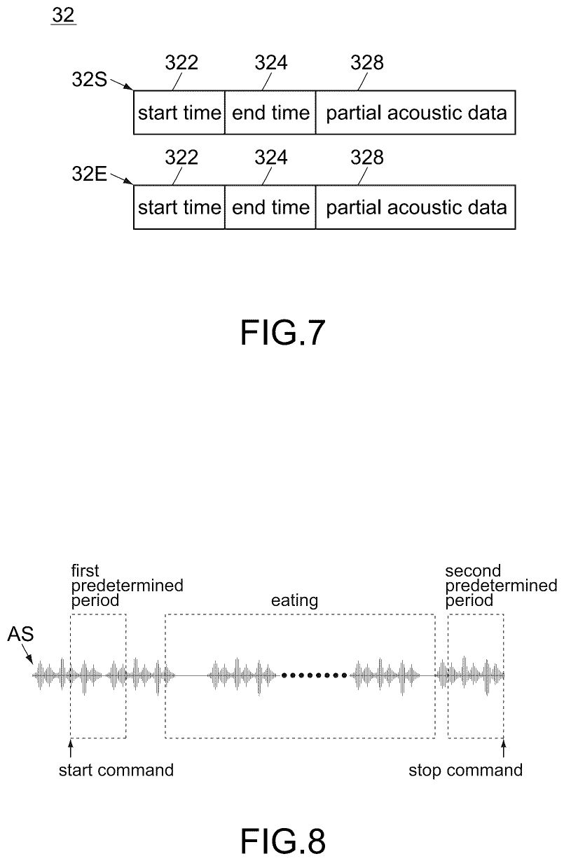

Referring to FIG. 7 together with FIG. 6, the acoustic data 32 is a file which memorizes the acoustic signals AS received by the device 20. The acoustic data 32 of the present embodiment is created when the analyzer 30 starts its process. The acoustic data 32 of the present embodiment is deleted before the analyzer 30 ends its process. Accordingly, the acoustic data 32 may have a constant name as its file name. However, the present invention is not limited thereto. For example, the analyzer 30 does not need to delete the acoustic data 32. In other words, the acoustic data 32 may be added every time the analyzer 30 starts its process. In this instance, the file name of the acoustic data 32 may be a combination of a constant name and a created date.

As described later, the acoustic data 32 is empty or null when created. The analyzer 30 stores a start record 32S and an end record 32E into the acoustic data 32 as the analyzer 30 performs processes. Each of the start record 32S and the end record 32E includes an acoustic data piece 328, a start time 322 and an end time 324. Each of the acoustic data pieces 328 is a part of the acoustic signals AS received by the device 20. Each of the start times 322 is a start time of the memorized acoustic data piece 328. Each of the end times 324 is an end time of the memorized acoustic data piece 328. The acoustic data 32 of the present embodiment has the aforementioned data structure. However, the present invention is not limited thereto. The data structure of the acoustic data 32 can be modified as necessary. Moreover, the acoustic data 32 may be provided as necessary.

Hereafter, explanation will be made about the analyzer 30 of the present embodiment.

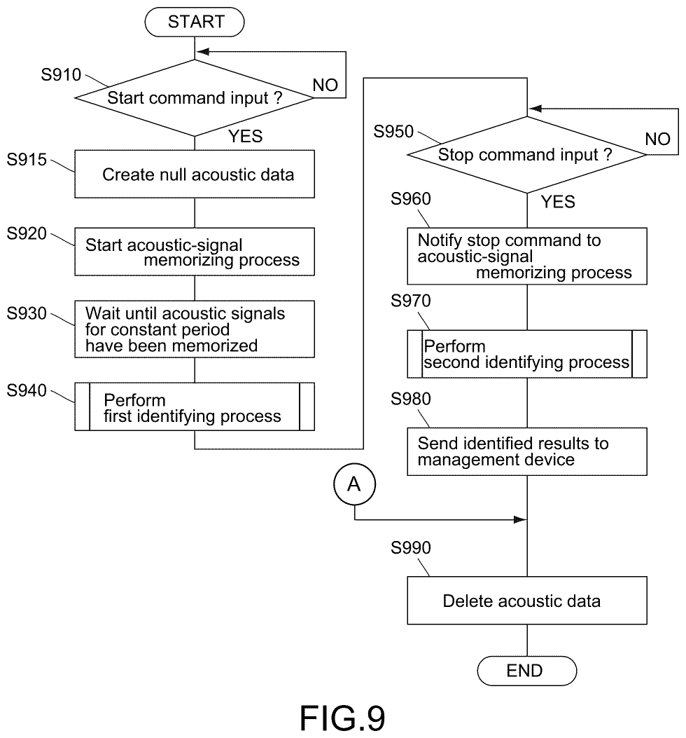

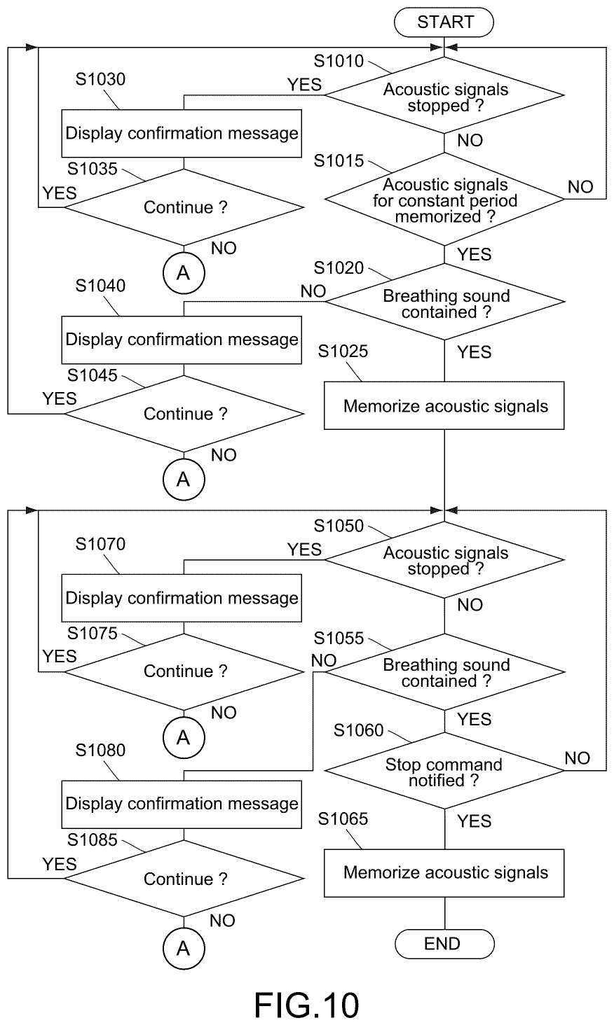

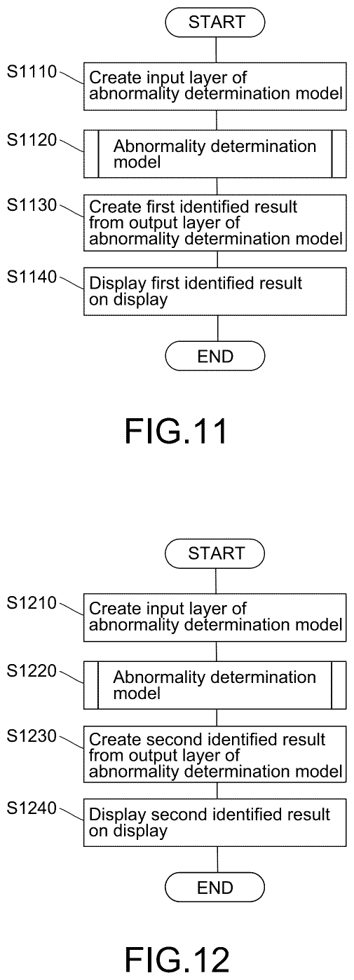

The analyzer 30 of the present embodiment performs four processes including a control process (see FIG. 9), an acoustic-signal memorizing process (see FIG. 10), the first identifying process (see FIG. 11) and the second identifying process (see FIG. 12). For example, the control process, the first identifying process and the second identifying process are executed on a thread, and the acoustic-signal memorizing process is executed on another thread. The performed acoustic-signal memorizing process continues to accumulatively memorize the acoustic signals AS received by the antenna 28 into the memory 22 until the second identifying process starts its process. However, the present invention is not limited thereto. The structure and the algorism of the analyzer 30 can be modified as necessary.

Hereafter, explanation will be made about the control process of the present embodiment.

Referring to FIG. 9 together with FIGS. 1, 6 and 7, after the start of the analyzer 30, the control process continues to wait until the operator input a start command (S910). For example, the control process displays a start button, or an input button indicating “start recording”, on the display 26 and continues to wait until the input button is pushed.

The control process creates null acoustic data 32, or the acoustic data 32 which includes no start record 32S and no end record 32E (S915), when the start command is input by the pushed input button (YES at S910). Then, the control process starts the acoustic-signal memorizing process (S920). As described later, the thus-started acoustic-signal memorizing process memorizes the start record 32S in the acoustic data 32 based on the received acoustic signals AS.

Then, the control process waits until the acoustic signals AS for a constant period CP have been memorized in the acoustic data 32 (S930). For example, the constant period CP is thirty seconds. More specifically, the control process waits until the acoustic-signal memorizing process stores the start record 32S. The control process performs the first identifying process (S940) when the start record 32S is stored. As described later, the first identifying process generates a first identified result which indicates whether the breathing sound memorized in the start record 32S is normal or abnormal. The first identified result is displayed on the display 26.

Referring to FIG. 8 together with FIG. 1, according to the present embodiment, the operator of the device 20 inputs the start command before the subject 70 starts eating. During the constant period CP just after the input of the start command, substantially no food remains in the mouth of the subject 70, and thereby abnormal breathing is not caused by eating.

Referring to FIG. 9 together with FIGS. 1, 6 and 7, then, the control process continues to wait until the operator inputs a stop command (S950). For example, the control process displays an end button, or an input button indicating “start analyzing” on the display 26, and continues to wait until the end button is pushed.

The control process notifies the stop command to the acoustic-signal memorizing process (S960) when the stop command is input by the pushed end button (YES at S950). As described later, the thus-notified acoustic-signal memorizing process memorizes the end record 32E in the acoustic data 32 based on the received acoustic signals AS.

Then, the control process performs the second identifying process (S970). As described later, the second identifying process generates a second identified result which indicates whether the breathing sound memorized in the end record 32E is normal or abnormal. The second identified result is displayed on the display 26.

Referring to FIG. 8 together with FIG. 1, according to the present embodiment, the operator of the device 20 inputs the stop command after the subject 70 ends eating. The operator may detach the acoustic sensor 60 from the subject 70 either before or after the input of the stop command.

Referring to FIG. 9 together with FIGS. 1, 6 and 7, then, the control process sends the identified results to the management device 40 (S980). The control process of the present embodiment sends both the first identified result and the second identified result depending on a combination of the first identified result and the second identified result. For example, the control process sends both the first identified result and the second identified result only under a condition where the first identified result is normal and the second identified result is abnormal. The operator of the management device 40 who receives these results can be aware that abnormal breathing is caused by the eating and thereby can provide necessary care for the subject 70.

Then, the control process deletes the acoustic data 32 (S990) and ends its process.

Hereafter, explanation will be made about the acoustic-signal memorizing process of the present embodiment.

Referring to FIG. 10 together with FIGS. 1, 6 and 7, the started acoustic-signal memorizing process continues to memorize the received acoustic signals AS into the memory 22 until receiving the acoustic signals AS for the constant period CP (S1015). The acoustic-signal memorizing process continues the aforementioned process while determining whether the acoustic signals AS are stopped or not (S1010). The acoustic-signal memorizing process determines whether the acoustic signals AS for the constant period CP have been received or not (S1015) in a case where the acoustic signals AS are not stopped, or the acoustic signals AS can be received (NO at S1010). The acoustic-signal memorizing process continues to receive the acoustic signals AS (S1010) in a case where the acoustic signals AS for the constant period CP have not been received (NO at S1015).

The acoustic-signal memorizing process determines whether a breathing sound is contained in the received acoustic signals AS or not (S1020) in a case where the acoustic signals AS for the constant period CP have been received (YES at S1015). The acoustic-signal memorizing process memorizes the received acoustic signals AS for the constant period CP into the start record 32S of the acoustic data 32 (S1025) in a case where a breathing sound is contained (YES at S1020).

In detail, the acoustic-signal memorizing process adds the start record 32S in the acoustic data 32. Meanwhile, the acoustic-signal memorizing process memorizes the start time 322, the end time 324 and the acoustic data piece 328 of the start record 32S. For the acoustic data piece 328, the acoustic-signal memorizing process memorizes the acoustic signals AS for the constant period CP. For the start time 322, the acoustic-signal memorizing process memorizes a time from which reception of the acoustic signals AS to be memorized begins. For the end time 324, the acoustic-signal memorizing process memorizes a time at which reception of the acoustic signals AS to be memorized comes to end.

The acoustic-signal memorizing process of the present embodiment determines that a breathing sound is contained in the acoustic signals AS received for the constant period CP in a case where the acoustic signals AS are intense. More specifically, the acoustic-signal memorizing process determines that a breathing sound is contained in a case where the amplitude of the received acoustic signals AS even partially exceeds a predetermined value. However, the present invention is not limited thereto. For example, the acoustic-signal memorizing process may determine whether a breathing sound is contained or not using a neural network model.

The acoustic-signal memorizing process continues to memorize the received acoustic signals AS into the memory 22 after the memorization of the acoustic signals AS into the acoustic data 32 until the stop command is notified from the control process (S1060). The acoustic-signal memorizing process continues the aforementioned process while determining whether the acoustic signals AS are stopped or not (S1050). The acoustic-signal memorizing process determines whether a breathing sound is contained or not in the acoustic signals AS received for the last constant period CP (S1055) at every predetermined unit period, for example, at every two seconds, in a case where the acoustic signals AS are not stopped (NO at S1050). The acoustic-signal memorizing process determines whether the stop command is notified from the control process or not (S1060) in a case where a breathing sound is contained (YES at S1055). The acoustic-signal memorizing process continues to receive the acoustic signals AS (S1050) in a case where the stop command is not notified (NO at S1060).

The acoustic-signal memorizing process memorizes the acoustic signals AS received during the last constant period CP into the end record 32E of the acoustic data 32 (S1065) and ends its process in a case where the stop command is notified from the control process (YES at S1060).

The acoustic-signal memorizing process displays a confirmation message on the display 26 (S1030 or S1070) in a case where the acoustic signals AS are stopped (No at S1010 or S1050). For example, the acoustic-signal memorizing process displays a continuation button and an end button, which are input buttons for selecting whether the process should be continued or should be ended, on the display 26 together with a message such as “check the sensor since no sound can be listened”.

The acoustic-signal memorizing process displays another confirmation message on the display 26 (S1040 or S1080) in a case where no breathing sound is contained in the received acoustic signals AS (NO at S1020 or S1055). For example, the acoustic-signal memorizing process displays a continuation button and an end button, which are input buttons for selecting whether the process should be continued or should be ended, on the display 26 together with a message such as “check the sensor since no breathing sound can be listened”.

The operator of the device 20 can be aware that some problems occurred on the acoustic sensor 60 by the aforementioned confirmation messages. For example, if the switch 63 (see FIG. 2) of the acoustic sensor 60 takes the off-state, the operator can push the switch 63 so that the switch 63 takes the on-state. If the acoustic sensor 60 is removed, the operator can attach the acoustic sensor 60 to the subject 70 again. Moreover, the operator can push the continuation button or the end button depending on the situation.

The acoustic-signal memorizing process continues to receive the acoustic signals AS again (S1010 or S1050) in a case where the continuation button is pushed (YES at S1035, S1045, S1075 or S1085). The acoustic-signal memorizing process throws an error to the control process and ends its process (A) in a case where the end button is pushed (NO at S1035, S1045, S1075 or S1085). Referring to FIG. 9 together with FIG. 6, the control process deletes the acoustic data 32 (S990) and ends its process when the error is thrown (A).

Referring to FIGS. 6 and 7, as can be seen from the explanation described above, the start record 32S of the acoustic data 32 memorizes the acoustic signals AS received during the constant period CP after the input of the start command. The end record 32E of the acoustic data 32 memorizes the acoustic signals AS received during the constant period CP before the input of the stop command. According to the present embodiment, necessary acoustic signals AS can be memorized in the acoustic data 32 by the acoustic-signal memorizing process which is relatively simple. Accordingly, the analyzer 30 can easily identify a breathing sound.

However, the present invention is not limited thereto, but the acoustic-signal memorizing process can be modified as necessary. For example, the acoustic-signal memorizing process may memorize all the acoustic signals AS received between the input of the start command and the input of the stop command into the acoustic data 32 for every constant period CP.

Hereafter, explanation will be made about the first identifying process and the second identifying process of the present embodiment.

Referring to FIG. 11 together with FIGS. 6 and 7, the first identifying process firstly creates an input layer of an abnormality determination model (S1110). As described later, the abnormality determination model is a neural network model which is generated using a machine-learning. More specifically, the first identifying process creates the input layer of the abnormality determination model based on the acoustic data piece 328, or the acoustic signals AS for the constant period CP, of the start record 32S stored in the acoustic data 32. Then, the first identifying process gets an output layer of the abnormality determination model using the created input layer (S1120). Then, the first identifying process creates the first identified result from the output layer of the abnormality determination model (S1130). The first identified result is an indicator which indicates whether a breathing sound contained in the acoustic signals AS during the constant period CP is normal or abnormal. Then, the first identifying process displays the first identified result on the display 26 (S1140).

Referring to FIG. 1, the first identified result of the present embodiment is displayed as “normal” or “abnormal”. The operator of the device 20 can provide necessary care for the subject 70 when “abnormal” is displayed. However, the present invention is not limited thereto. For example, the first identified result may be displayed as “O” or “X”.

Referring to FIG. 12 together with FIGS. 6 and 7, the second identifying process firstly creates the input layer of the abnormality determination model (S1210). More specifically, the second identifying process creates the input layer of the abnormality determination model based on the acoustic data piece 328, or the acoustic signals AS for the constant period CP, of the end record 32E stored in the acoustic data 32. Then, the second identifying process gets the output layer of the abnormality determination model using the created input layer (S1220). Then the second identifying process creates the second identified result from the output layer of the abnormality determination model (S1230). The second identified result is an indicator which indicates whether a breathing sound contained in the acoustic signals AS during the constant period CP is normal or abnormal. Then, the second identifying process displays the second identified result on the display 26 (S1240).

Referring to FIG. 1, the second identified result of the present embodiment is displayed as “normal” or “abnormal”. The operator of the device 20 can provide necessary care for the subject 70 when “abnormal” is displayed. However, the present invention is not limited thereto. For example, the second identified result may be displayed as “O” or “X”.

Summarizing the explanation about the device 20 described above with reference to FIGS. 1, 6 and 8, the device 20 of the present embodiment performs the first identifying process upon the start command and performs the second identifying process upon the stop command subsequent to the start command. In other words, the instructions stored in the memory 22, when executed by the processor 21, cause the processor 21 to perform the first identifying process upon the start command and to perform the second identifying process upon the stop command subsequent to the start command. The first identified result is displayed on the display 26 in the first identifying process. The first identified result indicates whether a breathing sound obtained during a first predetermined period is normal or abnormal. The second identified result is displayed on the display 26 in the second identifying process. The second identified result indicates whether a breathing sound obtained during a second predetermined period is normal or abnormal.

As previously described, the analyzer 30 is installed in the auxiliary storage 23 so that the device 20 works as described above. The analyzer 30 is memorized in a non-transitory computer-readable storage medium. Thus, this non-transitory computer-readable storage medium stores instructions executable by the processor 21 of the computer system (device 20) which comprises the display 26 coupled to the processor 21. The instructions stored in the non-transitory computer-readable storage medium comprises instructions for performing the first identifying process upon the start command, performing the second identifying process upon the stop command subsequent to the start command, displaying the first identified result on the display 26 in the first identifying process, and displaying the second identified result on the display 26 in the second identifying process.

Each of the first predetermined period and the second predetermined period of the present embodiment is equal to the constant period CP of the acoustic-signal memorizing process (see FIG. 10). Thus, each of the first predetermined period and the second predetermined period of the present embodiment is thirty seconds. However, the present invention is not limited thereto. Each of the first predetermined period and the second predetermined period may be any period during which the acoustic sensor 60 can collect a breathing sound. For example, each of the first predetermined period and the second predetermined period may be shorter than the constant period CP of the acoustic-signal memorizing process. In other words, the constant period CP of the acoustic-signal memorizing process may be longer than each of the first predetermined period and the second predetermined period. Moreover, the first predetermined period and the second predetermined period may be different from each other.

The device 20 according to the present embodiment can display the two identified results including the first identified result for the first predetermined period and the second identified result for the second predetermined period subsequent to the first predetermined period. Accordingly, two identified results before and after an event such as eating can be compared with each other by inputting the start command before the event and inputting the stop command after the event. It can be considered that abnormality of the breathing sound indicated by the second identified result occurs because of the event by comparing the identified results with each other.

More specifically, it can be considered that abnormal breathing occurs because of eating if the identified result before the eating of the subject 70 is normal and the identified result after the eating of the subject 70 is abnormal. For example, it can be considered that a piece of food might remain in an upper respiratory organ of the subject 70 because of the eating. Thus, the present embodiment provides the device 20 configured to detect abnormal breathing caused by events such as eating.

The event of the present embodiment is eating. However, the present invention is not limited thereto. For example, the event may be exercise, bathing or taking medicine such as inhalants for asthma. The first identifying process may be performed before one of these events, and the second identifying process may be performed after the one of these events. When the device 20 of the present embodiment is used for such events, it is possible not only to detect abnormal breathing generated because of the event but also to detect elimination of the abnormal breathing as a result of the event such as light exercise or taking medicine.

Referring to FIG. 10 together with FIG. 6, as previously described, the acoustic-signal memorizing process displays a confirmation message on the display 26 in a case where the acoustic signals AS cannot be received during the constant period CP. The acoustic-signal memorizing process displays another confirmation message on the display 26 in another case where a breathing sound is not contained in the received acoustic signals AS. In other words, the instructions stored in the memory 22, when executed by the processor 21, cause the processor 21 to display a message on the display 26 in a case where no breathing sound is obtained during the constant period CP after the first identifying process and before the stop command, the message showing that no breathing sound is not obtained. Moreover, the acoustic-signal memorizing process is performed on a thread other than that of the control process (see FIG. 9), the first identifying process (see FIG. 11) and the second identifying process (see FIG. 12). Accordingly, the acoustic-signal memorizing process can display a confirmation message on the display 26 at various timings.

For example, the acoustic-signal memorizing process can display a confirmation message on the display 26 even at a first timing, or before the first identifying process, and even at a second timing, or before the second identifying process. The acoustic-signal memorizing process can change the content of the confirmation message depending on the timing. For example, the device 20 can display a message, which indicates that a breathing sound is not obtained, on the display 26 in a case where a breathing sound is not obtained for the constant period CP after the first identifying process and before the stop command. The operator of the device 20 can be timely aware of various problems such as trouble of the acoustic sensor 60 by such a message.

The device 20 of the present embodiment can be further variously modified in addition to the already described various modifications. Hereafter, explanation will be made about modifications of the device 20.

Referring to FIG. 1, according to the present embodiment, each of the first identified result and the second identified result is displayed on the display 26. However, the present invention is not limited thereto. For example, the device 20 may use a speaker to make a sound in a case where the second identified result is abnormal.

Referring to FIG. 8, the first predetermined period of the present embodiment is a continuous period right after the input of the start command by the operator of the device 20. However, the present invention is not limited thereto. For example, the first predetermined period may be a discontinuous period which begins a few seconds after the input of the start command by the operator. The first predetermined period may be the total time of two or more of the thus-separated periods. Thus, the first predetermined period may be a period after the input of the start command. Referring to FIG. 9 together with FIG. 6, the analyzer 30 may start the acoustic-signal memorizing process (S920) right after its start, or before S910. Referring to FIG. 8, in this instance, the first predetermined period may include a period before the input of the start command by the operator or may be a period only before the input of the start command by the operator.

The second predetermined period of the present embodiment is a continuous period right before the input of the stop command by the operator. However, the present invention is not limited thereto. For example, the second predetermined period may be a discontinuous period which comes to end a few seconds before the input of the stop command by the operator. The second predetermined period may be the total time of two or more of the thus-separated periods. Thus, the second predetermined period may be a period before the input of the stop command. Moreover, the second predetermined period may include a period after the input of the stop command by the operator or may be a period only after the input of the stop command by the operator.

Referring to FIG. 1, as previously described, the device 20 of the present embodiment after the second identifying process sends the first identified result and the second identified result to the management device 40, which is different and distinct from the device 20, depending on a combination of the first identified result and the second identified result. In other words, the instructions stored in the memory 22 (see FIG. 6), when executed by the processor 21 (see FIG. 6), cause the processor 21, after the second identifying process, to send the first identified result and the second identified result to the management device 40 depending on a combination of the first identified result and the second identified result. However, the present invention is not limited thereto. For example, the device 20 may send the first identified result and the second identified result to the management device 40 regardless of the content of each of the first identified result and the second identified result. The device 20 may send none of the first identified result and the second identified result to the management device 40.

The device 20 of the present embodiment identifies a breathing sound only in the first identifying process and the second identifying process. Thus, the device 20 of the present embodiment enables to obtain the two identified result only for the first predetermined period and the second predetermined period which are separated from each other with the event occurring therebetween. However, the present invention is not limited thereto. For example, the device 20 may identify a breathing sound generated in a third predetermined period during an event in addition to the first identifying process and the second identifying process.

Referring to FIGS. 11 and 12, the device 20 of the present embodiment identifies a breathing sound as normal or abnormal using the abnormality determination model. The abnormality determination model of the present embodiment is a neural network model which is generated via machine-learning, or deep learning. According to the present embodiment, a breathing sound can be identified as normal or abnormal with an accuracy of 80% or more, for example. However, the present invention is not limited thereto. For example, a method for identifying a breathing sound as normal or abnormal is not specifically limited.

Hereafter, explanation will be made about the abnormality determination model (see FIG. 11) of the present embodiment.

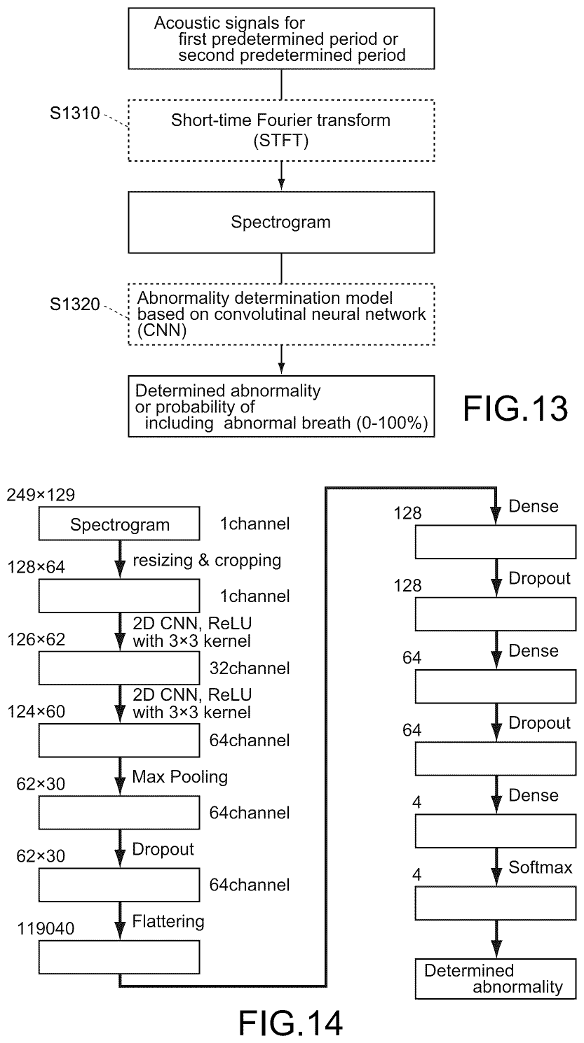

Referring to FIG. 13 together with FIGS. 6, 11 and 12, the device 20 generates a spectrogram by applying Short-time Fourier transform (STFT) to the acoustic signals AS during the first predetermined period or the second predetermined period (S1110 of FIG. 11, S1210 of FIGS. 12 and S1310 of FIG. 13). The device 20 gets the output layer of the abnormality determination model using the generated spectrogram as the input layer (S1130 of FIG. 11, S1230 of FIGS. 12 and S1320 of FIG. 13).

The spectrogram of the present embodiment has a size of 249×129 pixels and 1 channel. The abnormality determination model of the present embodiment is a convolutional neural network (CNN) model. For example, the abnormality determination model of the present embodiment is a machine-learned neural network model having a layer structure illustrated in FIG. 14. The output layer of the abnormality determination model contains probability that the breathing sound is abnormal. More specifically, the output layer contains value between 0% to 100% (both inclusive). However, the present invention is not limited thereto. For example, the abnormality determination model may be any type of model. The input layer and the output layer of the abnormality determination model can be modified as necessary. For example, the input layer may contain a reversed spectrogram.

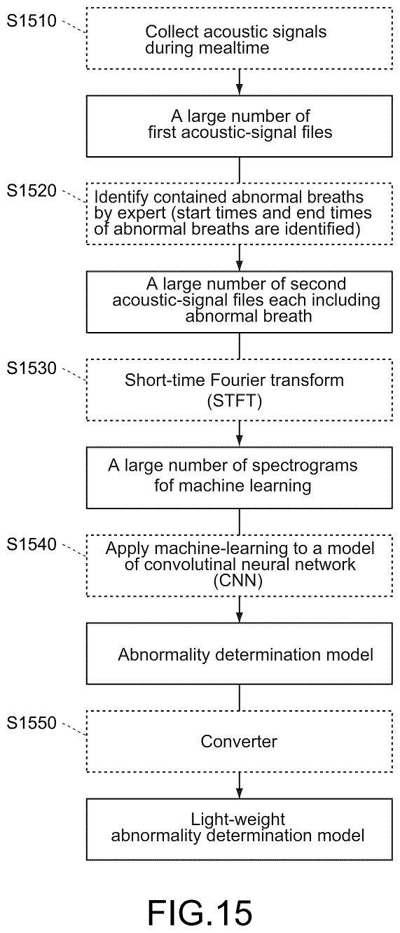

The abnormality determination model of the present embodiment can be generated as shown in FIG. 15, for example.

Firstly, acoustic signals AS during mealtime are collected so that a large number of first acoustic-signal files are created (S1510). Then, an expert such as a speech therapist listens to the first acoustic-signal files and extracts abnormal breathings (S1520). More specifically, a start time and an end time of each of the abnormal breathing sounds are memorized in each of the first acoustic-signal files. A large number of second acoustic-signal files are created based on the thus-memorized start times and end times. Each of the second acoustic-signal files includes a part of the acoustic signals AS during which abnormal breathing is generated. Then, STFT is applied to each of the second acoustic-signal files so that a large number of spectrograms for machine-learning are generated (S1530). Then, a machine-learning is performed using the generated spectrograms as teaching data (S1540). The abnormality determination model is generated as a result of this machine-learning. Then, the generated abnormality determination model is converted by a converter into a light-weight abnormality determination model which can be installed in a mobile terminal.

The abnormality determination model of the present embodiment can be generated as described above. However, the present invention is not limited thereto. For example, a generation method of the abnormality determination model is not specifically limited.

For example, an abnormality determination model which continues to learn may be installed in a mobile terminal instead of the light-weight abnormality determination model which does not learn. Referring to FIG. 6, the analyzer 30 according to this instance may leave the acoustic data 32 as it is without deleting it when ending its process. Thus, the acoustic data 32 may be accumulatively stored. The accuracy of identification of the abnormality determination model can be improved by a machine-learning which uses a large amount of the accumulatively stored acoustic data 32 to identify a breathing sound as normal or abnormal.

The system 10 (see FIG. 1) of the present embodiment can be modified as described below.

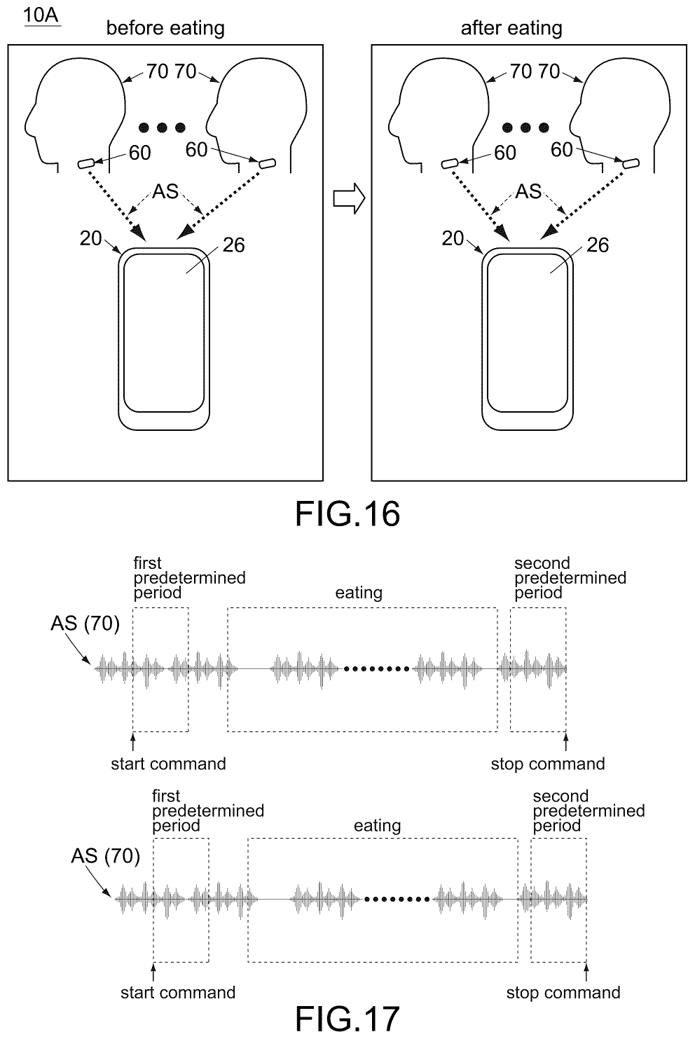

Referring to FIG. 16, a system 10A for identifying a breathing sound according to a modification comprises the device 20 and two or more of the acoustic sensors 60. The acoustic sensors 60 are attached to two or more of the subjects 70, respectively, via adhering. Each of the acoustic sensors 60 in operation is configured to collect surrounding sounds, convert them into the acoustic signals AS and send out the acoustic signals AS. The device 20 is paired with each of the acoustic sensors 60. The device 20 is configured to receive the acoustic signals AS sent from two or more of the acoustic sensors 60.

Referring to FIG. 16 together with FIG. 17, the device 20 of the present modification performs the control process (see FIG. 9), the acoustic-signal memorizing process (see FIG. 10), the first identifying process (see FIG. 11) and the second identifying process (see FIG. 12) for each of the paired acoustic sensors 60. For example, the device 20 can work for two or more of the acoustic sensors 60 in parallel to each other. The two or more of the acoustic sensors 60 may be selected from a list of sensors (not shown) displayed on the display 26. Thus, the device 20 of the present modification can perform the first identifying process for two or more sets of the acoustic signals AS, the first predetermined periods of the two or more sets of the acoustic signals AS overlapping with each other, each of the two or more sets of the acoustic signals AS containing a breathing sound. The device 20 of the present modification can also perform the second identifying process for two or more sets of the acoustic signals AS, the second predetermined periods of the two or more sets of the acoustic signals AS overlapping with each other, each of the two or more sets of the acoustic signals AS containing a breathing sound. In other words, the instructions stored in the memory 22 (see FIG. 6), when executed by the processor 21 (see FIG. 6), cause the processor 21 to perform the first identifying process for the two or more sets of the acoustic signals AS and to perform the second identifying process for the two or more sets of the acoustic signals AS.

When the device 20 of the present modification is paired with each of the acoustic sensors 60, the device 20 may read the two-dimensional matrix barcode of the tag 69 (see FIG. 2) attached on the base 67 (see FIG. 2) and may memorize the identifier of the acoustic sensor 60 in relation with the acoustic sensor 60. For example, the thus-memorized identifiers enable the device 20 to display the identifier of the acoustic sensor 60 on the display 26 together with the first identified result.

According to the present modification, abnormal breathing of two or more of the subjects 70 can be detected by the single device 20. However, the system 10A may comprise two or more of the devices 20 and two or more of the acoustic sensors 60. In this instance, two or more of the acoustic sensors 60 may be paired with two or more of the devices 20, respectively, or may be paired with the same device 20. In an instance in which two or more of the devices 20 are paired with the same acoustic sensor 60, the two or more of the devices 20 can detect abnormal breathing of one of the subjects 70. Accordingly, the possibility of missing abnormal breathing can be reduced.

As can be seen from the explanation described above, the present modification provides a method for watching over two or more of the subjects 70 using the system 10A. According to this watching method, the acoustic sensors 60 are attached to the subjects 70, respectively. The acoustic sensors 60 are started and the start command is input to each of the devices 20 before all the subjects 70 start a predetermined event such as eating. The stop command is input to each of the devices 20 after all the subjects 70 end the predetermined event. This watching method enables efficient care for the subjects 70.

While there has been described what is believed to be the preferred embodiment of the invention, those skilled in the art will recognize that other and further modifications may be made thereto without departing from the spirit of the invention, and it is intended to claim all such embodiments that fall within the true scope of the invention.

What is claimed is:

1. A device for identifying a breathing sound contained in acoustic signals received from outside, the device comprising a processor, a display coupled to the processor and a memory coupled to the processor and storing instructions, wherein the instructions, when executed by the processor, cause the processor to:
perform a first identifying process upon a start command;
perform a second identifying process upon a stop command subsequent to the start command;
display a first identified result on the display in the first identifying process, the first identified result indicating whether a breathing sound obtained during a first predetermined period is normal or abnormal; and
display a second identified result on the display in the second identifying process, the second identified result indicating whether a breathing sound obtained during a second predetermined period is normal or abnormal.

2. The device as recited in claim 1, wherein the instructions, when executed by the processor, cause the processor, after the second identifying process, to send the first identified result and the second identified result to a management device depending on a combination of the first identified result and the second identified result, the management device being different and distinct from the device for identifying a breathing sound.

3. The device as recited in claim 1, wherein:
the device is a mobile terminal; and
the display is a screen of the mobile terminal.

4. The device as recited in claim 1, wherein the instructions, when executed by the processor, cause the processor to display a message on the display in a case where no breathing sound is obtained during a constant period after the first identifying process and before the stop command, the message showing that no breathing sound is not obtained.

5. The device as recited in claim 2, wherein the instructions, when executed by the processor, cause the processor to display a message on the display in a case where no breathing sound are obtained during a predetermined period after the first identifying process and before the stop command, the message showing that no breathing sound is not obtained.

6. The device as recited in claim 3, wherein the instructions, when executed by the processor, cause the processor to display a message on the display in a case where no breathing sound are obtained during a predetermined period after the first identifying process and before the stop command, the message showing that no breathing sound is not obtained.

7. The device as recited in claim 1, wherein:
the instructions, when executed by the processor, cause the processor to:
perform the first identifying process for two or more sets of the acoustic signals, the first predetermined periods of the two or more sets of the acoustic signals overlapping with each other, each of the two or more sets of the acoustic signals containing the breathing sound; and
perform the second identifying process for two or more sets of the acoustic signals, the second predetermined periods of the two or more sets of the acoustic signals overlapping with each other, each of the two or more sets of the acoustic signals containing the breathing sound.

8. The device as recited in claim 2, wherein:
the instructions, when executed by the processor, cause the processor to:
perform the first identifying process for two or more sets of the acoustic signals, the first predetermined periods of the two or more sets of the acoustic signals overlapping with each other, each of the two or more sets of the acoustic signals containing the breathing sound; and
perform the second identifying process for two or more sets of the acoustic signals, the second predetermined periods of the two or more sets of the acoustic signals overlapping with each other, each of the two or more sets of the acoustic signals containing the breathing sound.

9. The device as recited in claim 3, wherein:
the instructions, when executed by the processor, cause the processor to:
perform the first identifying process for two or more sets of the acoustic signals, the first predetermined periods of the two or more sets of the acoustic signals overlapping with each other, each of the two or more sets of the acoustic signals containing the breathing sound; and
perform the second identifying process for two or more sets of the acoustic signals, the second predetermined periods of the two or more sets of the acoustic signals overlapping with each other, each of the two or more sets of the acoustic signals containing the breathing sound.

10. A non-transitory computer-readable storage medium storing instructions executable by a processor of a computer system which comprises a display coupled to the processor, the instructions comprising instructions for:
performing a first identifying process upon a start command;
performing a second identifying process upon a stop command subsequent to the start command;
displaying a first identified result on the display in the first identifying process, the first identified result indicating whether a breathing sound obtained during a first predetermined period is normal or abnormal; and
displaying a second identified result on the display in the second identifying process, the second identified result indicating whether a breathing sound obtained during a second predetermined period is normal or abnormal.

11. A system comprising the device as recited in claim 1 and two or more of acoustic sensors, wherein:
each of the acoustic sensors in operation is configured to collect surrounding sounds, convert them into acoustic signals and send out the acoustic signals; and
the device is configured to receive the acoustic signals sent from the two or more of the acoustic sensors.

12. A system comprising the device as recited in claim 2 and two or more of acoustic sensors, wherein:
each of the acoustic sensors in operation is configured to collect surrounding sounds, convert them into acoustic signals and send out the acoustic signals; and
the device is configured to receive the acoustic signals sent from the two or more of the acoustic sensors.

13. A system comprising the device as recited in claim 3 and two or more of acoustic sensors, wherein:
each of the acoustic sensors in operation is configured to collect surrounding sounds, convert them into acoustic signals and send out the acoustic signals; and
the device is configured to receive the acoustic signals sent from the two or more of the acoustic sensors.
